(19) United States
(12) Patent Application Publication
(10) Pub. No.: US 2005/0034179 A1
(43) Pub. Date: Feb. 10, 2005

(54) Non-human mammal model of autoimmune disease having OX40L gene transferred thereinto

(76) Inventors: Kazuo Sugamura, Sendai-shi (JP); Kazuko Murata, Sendai-shi (JP)

Correspondence Address: FROMMER LAWRENCE & HAUG, 745 FIFTH AVENUE- 10TH FL., NEW YORK, NY, 10151, US

(21) Appl. No.: 10/810,527

(22) Filed: Mar. 26, 2004

Related U.S. Application Data
(63) Continuation-in-part of application No. PCT/JP02/07674, filed on Jul. 29, 2002.

(30) Foreign Application Priority Data
Sep. 28, 2001 (JP) 2001-304645

(51) Int. Cl. A01K 67/027

(52) U.S. Cl. 800/10; 800/18

(57) ABSTRACT

The present invention provides a transgenic non-human mammal model of autoimmune disease introduced with an OX40L gene, and a method for using said mammal for screening a therapeutic drug for the autoimmune disease. Transgenic mice wherein OX40L, one of the TNF family molecules, is constantly expressed in T cells, were constructed. These mice developed an autoimmune disease and they were found to be useful as a model of autoimmune disease. The transgenic non-human mammal that develops an autoimmune disease in the present invention can be constructed by expressing the OX40L gene under the control of T cell-specific lck promoter. The transgenic non-human mammal of the present invention develops an autoimmune disease such as interstitial pneumonia, inflammatory bowel disease, splenomegaly or lymphadenopathy, or hyperimmunoglobulinemia, and said mammal can be effectively used for screening a therapeutic drug for these autoimmune diseases.

### REFERENCE TO RELATED APPLICATIONS

The present application is a continuation-in-part of International Application PCT/JP02/07674 filed Jul. 29, 2002 and published as WO 03/028444 on Apr. 10, 2003, which claims priority to Japanese Patent Application 2001-304645 filed Sep. 28, 2001. Each of the above applications, and each document cited in this text and in each of the above applications (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### FIELD OF THE INVENTION

The present invention relates to a non-human mammal model of autoimmune disease, specifically, to a transgenic non-human mammal model of autoimmune disease introduced with an OX40L gene, and using said mammal for screening a therapeutic drug for an autoimmune disease.

### BACKGROUND

OX40 (CD134) is a molecule that belongs to the TNF receptor family, and transiently expresses in activated T cells. Meanwhile, OX40L (OX40 ligand) expresses in APCs (antigen presenting cells) such as activated B cells, activated dendritic cells and the like. There have been many reports regarding the effect of TNF family member when co-stimulated through the interaction of T-APC cells, and as its causation, the role they play is emphasized from the point of causation of autoimmune disease. In these reports, OX40L gene-deficient mice and OX40L transgenic mice that express OX40L in a limited way are constructed.

The following is a detailed explanation of the conventional reports regarding the effect of TNF family member when co-stimulated through the interaction of T-APC cells.

In order to sufficiently activate I T cells, not only the interaction of TCR (T cell receptor) with the peptide/MHC (major histocompatibility complex) complex, but also the co-stimulation by associate molecules that express on APCs (antigen presenting cells) are necessary. Aside from CD80 and CD86, both of which are known accessory molecules that bind to CD28 on T cells, several kinds of tumor necrosis factor (TNF) family members including OX40L (OX40 ligand), CD70, 4-1BBL and RRANCE are known to induce co-stimulation signal when binding to the receptor of the same system on the T cell. Further, it is known that CD40L, which is a different member that expresses on the T cell, is also indispensable for the activation of APC when binding to a receptor on APC (Grewal and Flavell, 1998, Annu. Rev. Immunol., 16, 111).

These observation results suggest that the TNF/TNF receptor family member play a role of co-stimulating in the T-APC interaction.

Originally, OX40L (Miura et al., 1991, Mol. Cell. Biol., 11, 1313), a molecule that was identified as human gp34 wherein its expression is induced by Tax of human T cell leukemia virus type I (HTLV-I), was thought to express in B cells, dendritic cells and endothelial cells (Ohshima et al., J. Immunol. 1997 Oct. 15; 159 (8): 3838-48; Kawamata et al., 1998; Murata et al., 2000, J. Exp. Med., 191, 365), and that its receptor, OX40, is basically an activated T cell marker (Paterson et al., 1987; Mallet et al., 1990). Evidences showing that the OX40L/OX40 interaction between T cells and APCs is greatly involved in the optimum CD4⁺ T cell reaction have come out continuously.

The present inventors cooperated with other researchers to construct OX40L-deficient mice. The said mice showed great deficiency of APC function when given an antigen, and due to this deficiency, T cell reaction in the production and proliferation for both Th1 and Th2 cytokine decreased (Chen et al., 1999, Immunity, 11, 689; Pippig et al., 1999, J. Immunol., 163, 652; Murata et al., 2000, J. Exp. Med., 191, 365). The deficiency of the same APC function can also be induced by interruption of OX40L/OX40 interaction in vivo using an anti-OX40L mAbs (monoclonal antibody) (Murata et al., 2000, J. Exp. Med., 191, 365). It is also known that the CD4⁺ T cell function decreases in mice deficient in OX40L and its receptor OX40 (Kopf et al., Immunity., 1999 December; 11 (6): 699-708). It is also shown that aside from the strong co-stimulation to both Th1 and Th2 reaction, OX40L/OX40 interaction also suppresses the reaction biased to Th2 under a certain experiment condition (Flynn et al., J. Exp. Med. 1998 Jul. 20; 188 (2): 297-304; Oshima et al., 1998; Jember et al., J. Exp. Med. 2001 Feb. 5; 193 (3): 387-92), when changing the antibody reaction (Chen et al., 1999, Immunity, 11, 689; Pippig et al., 1999, J. Immunol., 163, 652; Murata et al., 2000, J. Exp. Med., 191, 365; Morimoto et al., J. Immunol. 2000 Apr. 15; 164 (8): 4097-104), and during migration of T cells (Higgins et al., 1999, J. Immunol., 162, 486; Nohara et al., J. Immunol. 2001 Feb. 1; 166 (3): 2108-15).

OX40/OX40L is important in the development of memory T cells and regulation of survival of cells (Gramaglia et al., 1998, J. Immunol., 161, 6510 and 2000, J. Immunol., 165, 3043). Recently, the present inventors showed that when adoptive transfer of encephalitis-inducible wild-type T cells into OX40L-deficient deficient mice is conducted, persistent progress of the disease condition cannot be conducted in those mice (Ndhlovu et al., 2001, J. Immunol., 167, 2991). Further, there is a report that the stimulation of OX40 interrupts tolerance induction (Pakala et al., Nat. Med. 2001 August; 7 (8): 907-12), and this indicates the possibility that the OX40L/OX40 system is involved in the suppression of autoimmune disease. Certainly, OX40 and OX40L are detected in tissues of several kinds of inflammatory disorder such as experimental allergic encephalitis (EAE), allogenic graft versus host disease (GVH disease: GVHD), proliferative lupus nephritis and arthritis, etc. (Weinberg et al., 1999, J. Immunol., 162, 1818; Tittle et al., 1997, Blood, 89, 4652; Stüber et al., 1998, Gastroenterology, 115, 1205; Nakajima et al., J. Immunol. 2001 Feb. 1; 166 (3): 2108-15).

Autoreactive T cells that express OX40 were detected from rats suffering from EAE (experimental allergic encephalitis, and when OX40 antitoxin was administered, the symptom of EAE improved. In addition, it is thought that the soluble OX40 fusion protein, an antagonist of OX40L, not only suppresses the EAE (Weinberg et al., 1999, J. Immunol., 162, 1818) and GVHD (Stüber et al., 1998, Gastroenterology, 115, 1205) in progress, but also improves the symptoms of colitis (Higgins et al., 1999, J. Immunol., 162, 486), asthma (Jember et al., J Exp. Med. 2001 Feb. 5; 193 (3): 387-92), collagen induced arthritis (CIA) (Nakajima et al., J. Immunol. 2001 Feb. 1; 166 (3): 2108-15) in progress, in a mouse model of inflammatory bowel disease (IBD). These data suggest that the OX40L-OX40 interaction plays an important role in the immunoregulation of various autoimmune diseases.

The present inventors obtained a method for determining in more detail the severity of the effect of OX40L to immunoregulation, by excessive OX40L signaling that is irrelevant to the expression of OX40L on APC. Lane et al. constructed OX40L transgenic (OX40L-Tg) mice under the CD11c promoter, however, since the expression of OX40 was limited, they could not clearly indicate the functional significance of OX40 stimulation in T cell function. The expression of OX40L cannot be immediately detected necessarily on normal activated T cells (Murata et al., 2000, J. Exp. Med., 191, 365). As it was detected by immunostaining, it has been recently revealed by the present inventors that normal human T cell clones cultured for a long period of time express OX40L (Takasawa et al., 2001, Jpn. J. Cancer Res., 92, 377). Further, it was revealed that activated T cells derived from OX40-deficient mice can express OX40L on the cell surface (Kopf et al., Immunity. 1999 December; 11 (6): 699-708), which indicates the possibility of T cells expressing OX40L.

The object of the present invention is to provide a non-human mammal model of autoimmune disease and its use, more specifically, a transgenic non-human mammal model of autoimmune disease introduced with an OX40L gene, and using said mammal for screening a therapeutic drug for the autoimmune disease.

In a research regarding the effect of TNF family member when co-stimulated through the interaction of T-APC cells, the present inventors have constructed transgenic mice that constantly express OX40L (OX40 ligand), one of the TNF family molecules in the T cells, and have found out that these mice develop autoimmune disease and is useful as a model of autoimmune disease, thus the present invention has been completed.

The transgenic non-human mammal that develops autoimmune disease of the present invention can be constructed by introducing an OX40L gene into a fertilized egg of the non-human mammal by using an expression plasmid DNA that is constituted by an OX40L cDNA under the control of the T cell-specific lck promoter. The transgenic non-human mammal of the present invention develops autoimmune disease such as interstitial pneumonia, inflammatory bowel disease, splenomegaly or lymphadenopathy, or hyperimmunoglobulinemia, and said mammal can be effectively used for screening a therapeutic drug for these autoimmune diseases.

### SUMMARY OF THE INVENTION

The present invention relates to a transgenic non-human mammal introduced with an OX40L gene and constantly expresses OX40L in T cells (paragraph 1); the transgenic non-human mammal according to paragraph 1, wherein the OX40L gene is comprised of a DNA sequence of GenBank Accession No. U12763 (SEQ ID NO: 1) (paragraph 2); the transgenic non-human mammal according to paragraph 1 or 2, wherein the OX40L gene is introduced under the control of a T cell-specific lck promoter (paragraph 3); the transgenic non-human mammal according to any one of paragraphs 1 to 3 introduced with the OX40L gene and has an onset of an autoimmune disease (paragraph 4); the transgenic non-human mammal according to paragraph 4, wherein the autoimmune disease is an interstitial pneumonia (paragraph 5); the transgenic non-human mammal according to paragraph 4, wherein the autoimmune disease is an inflammatory bowel disease (paragraph 6); the transgenic non-human mammal according to paragraph 6, wherein the inflammatory bowel disease has an onset of moderate to severe hyperplasia of lymphatic system in the intestinal basal membrane, hyperplasia of mucous epithelium in basal membrane, lymphocyte invasion or hyperplasia of submucous lymphoid follicle (paragraph 7); the transgenic non-human mammal according to paragraph 4, wherein the autoimmune disease is a splenomegaly or lymphadenopathy (paragraph 8); the transgenic non-human mammal according to paragraph 4, wherein the autoimmune disease is a hyperimmunoglobulinemia (paragraph 9); and the transgenic non-human mammal according to any one of paragraphs 1 to 9, wherein the transgenic non-human mammal is a mouse (paragraph 10).

Further, the present invention relates to a method for constructing a transgenic non-human mammal, wherein an expression plasmid DNA that encodes OX40L is injected into a pronucleus of a fertilized egg of a non-human mammal to introduce an OX40L gene into a non-human mammal (paragraph 11); the method for constructing a transgenic non-human mammal according to paragraph 11, wherein the OX40L gene is comprised of a DNA sequence of GenBank Accession No. U12763 (paragraph 12); the method for constructing a transgenic non-human mammal according to paragraph 11 or 12, wherein the expression plasmid DNA that encodes OX40L is comprised of an OX40L cDNA under the control of a T cell-specific lck promoter (paragraph 13); the method for constructing a transgenic non-human mammal according to any one of paragraphs 11 to 13, wherein the transgenic non-human mammal introduced with an OX40L gene is purified by backcross (paragraph 14); the method for constructing a transgenic non-human mammal according to paragraph 14, wherein the backcross is conducted at least for 12 generations (paragraph 15); the method for constructing a transgenic non-human mammal according to any one of paragraphs 11 to 15, wherein the non-human mammal is a mouse (paragraph 16); and the method for constructing a transgenic non-human mammal according to paragraph 16, wherein an OX40L expression vector wherein an OX4OL gene is integrated downstream of the lck promoter, introduced into a mouse fertilized egg, and the mouse is backcrossed to a C57BL/6 line mouse (paragraph 17).

Still further, the present invention relates to a method for screening a therapeutic drug for an autoimmune disease that uses a transgenic non-human mammal according to any one of paragraphs 1 to 9 (paragraph 18); the method for screening a therapeutic drug for an autoimmune disease according to paragraph 18, wherein the autoimmune disease is interstitial pneumonia, inflammatory bowel disease, splenomegaly or lymphadenopathy, or hyperimmunoglobulinemia (paragraph 19); the method for screening a therapeutic drug for an autoimmune disease according to paragraph 17 or 18, wherein a test substance is administered to the transgenic non-human mammal according to any one of paragraphs 1 to 9, and changes in symptoms of autoimmune disease are evaluated and determined (paragraph 20); the method for screening a therapeutic drug for an autoimmune disease according to any one of paragraphs 18 to 20, wherein the transgenic non-human mammal is a mouse (paragraph 21); and a therapeutic drug for diabetes obtained from the method for screening a therapeutic drug for an autoimmune disease according to any one of paragraphs 18 to 21 (paragraph 22).

### BRIEF DESCRIPTION OF THE DRAWINGS

FIG. 1 is a view that shows the construct of OX40L in the OX40L transgenic mouse of the present invention, and its expression.

FIG. 1A. The construct of the expression vector of mouse OX40L is comprised as a mouse OX40L cDNA controlled by a T cell-specific lck promoter.

FIG. 1B. A view showing the results of the expression of OX40L in the thymus or spleen of the 3 individual first generation mice constructed or wild-type mice, by flow cytometry analysis. The small dotted lines show those derived from wild-type mice, the heavy lines show those derived from OX40L-Tg1 mice, the thin lines show those derived from OX40L-Tg2 mice, the large dotted lines show those derived from OX40L-Tg3 mice, respectively.

FIG. 2 is a view that shows the phenotype of the OX40L transgenic mice of the present invention.

FIG. 2A,. The total number of cells in the spleen of wild-type mice (□) and OX40LTg mice (▪), and the number of CD4⁺ T cells and CD8⁺ T cells, respectively.

FIG. 2B. The expression of CD25 and CD69 in the CD4⁺ T cells of the spleen.

FIG. 3 shows the memory T cell group and cytoplasmic cytokine in the OX4OL transgenic mouse of the present invention.

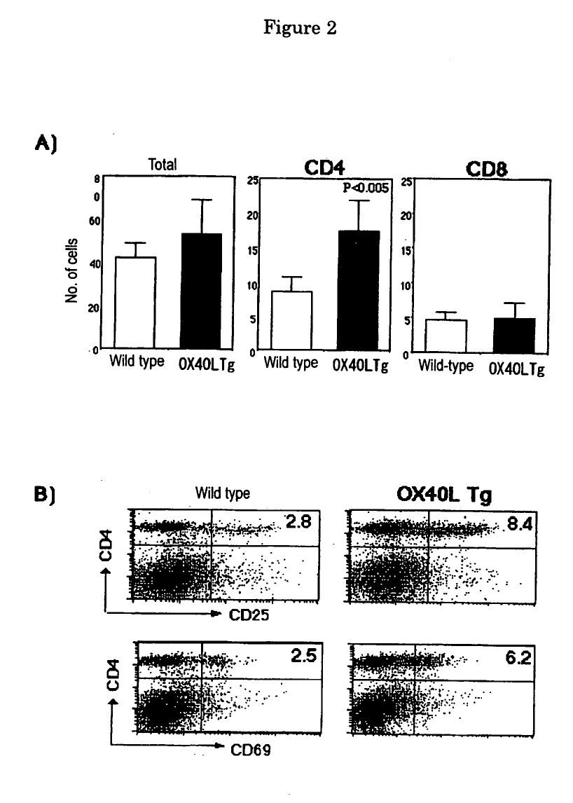

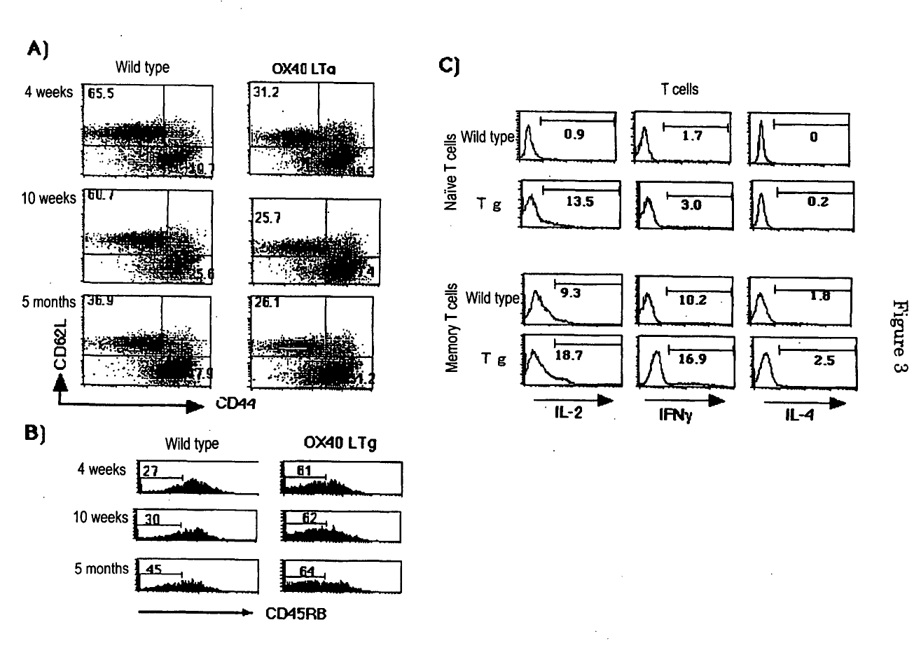

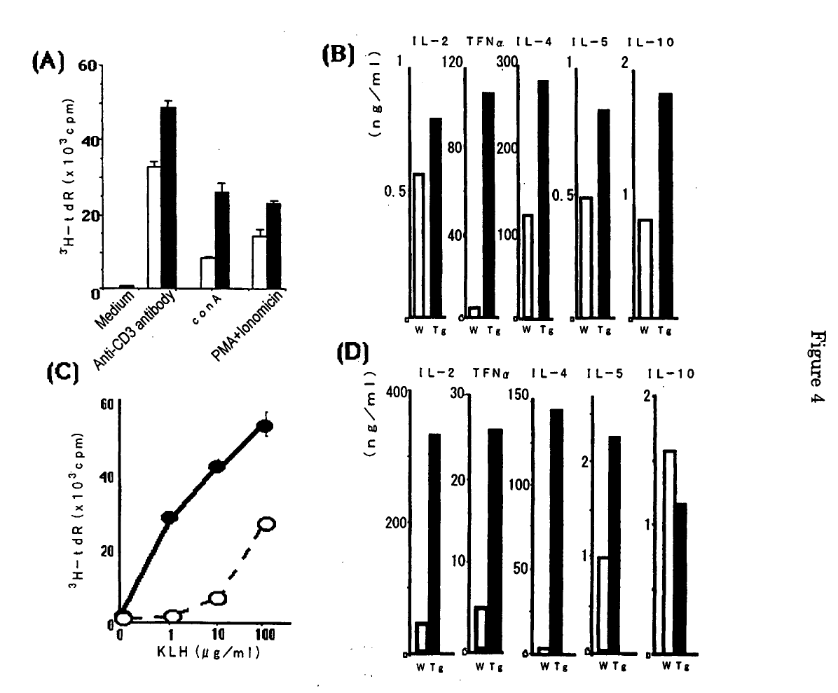

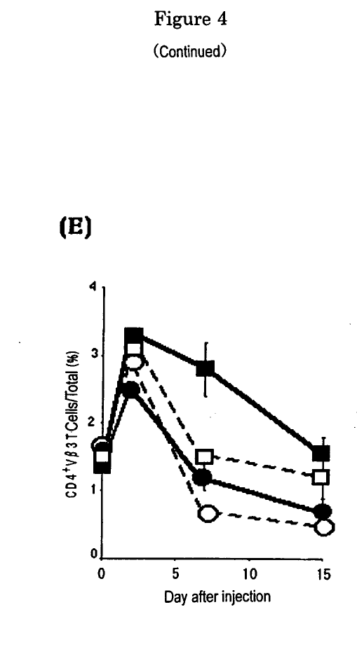

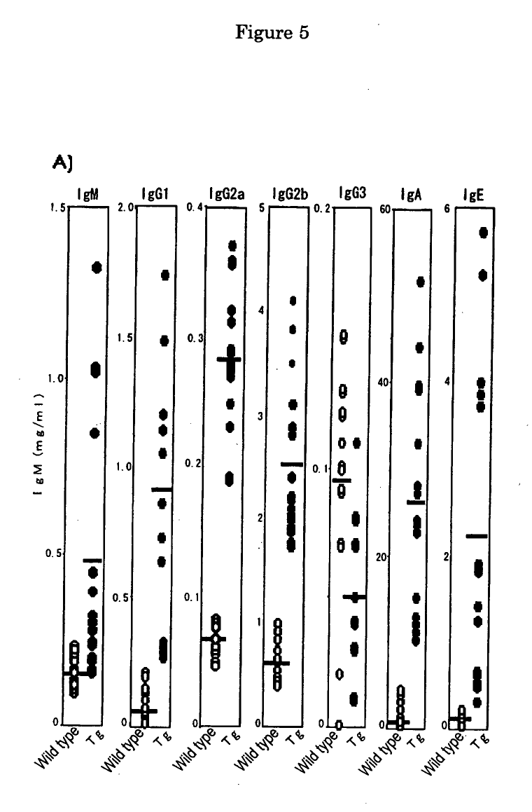

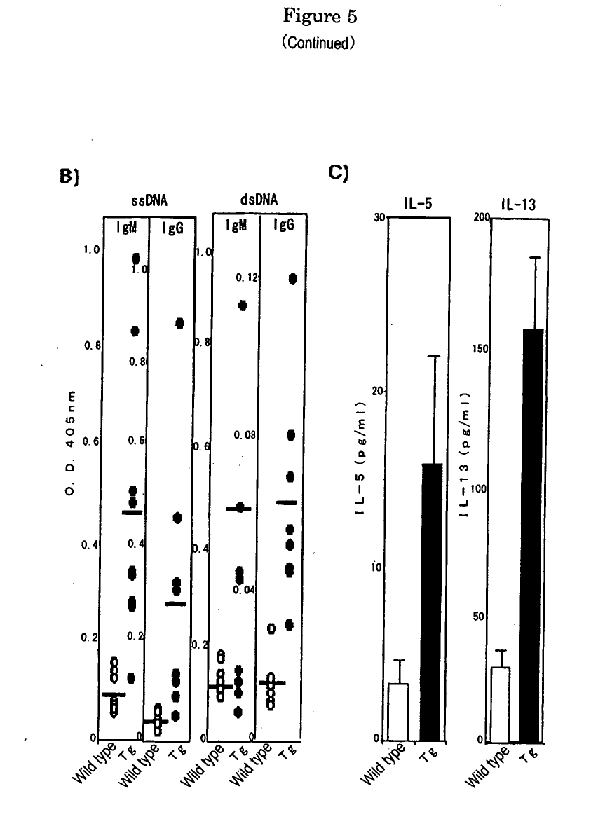

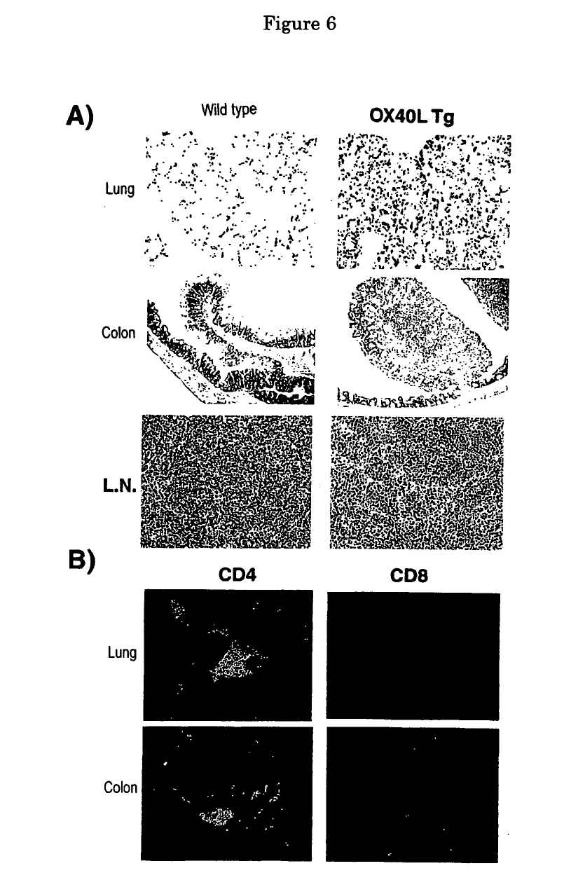

FIGS. 2A and 2B. The memory T cells showed a characteristic of increase in expression of CD44 and decrease in expression of CD62L and CD45RB.

FIG. 2C. Naive (CD62L^{high} CD44^{low}) CD4⁺ T cells and memory (CD62L^{low} CD44^{high}) CD4⁺ T cells were stimulated with PMA and ionomycin and then cytoplasm IL-2, IFNγ and IL-4 were detected by staining.

FIG. 4 is a view that shows the activation of proliferative T cells in the OX40L transgenic mice of the present invention.

FIG. 4A. The investigation results of proliferative reaction of spleen T cells derived from wild-type mice (□) and OX40L transgenic mice (▪) against phorbol myristate acetate (PMA) added with anti-CD3 antibody, concanavalin A (Con A) and ionomycin.

FIG. 4B. The investigation results of the amount of cytokine produced of the T cells stimulated by using an anti-CD3 antibody.

FIG. 4C. The antigen (KLH)-specific recall proliferation results of spleen T cells of wild-type mouse (□) or OX40L transgenic mice (▪).

FIG. 4D. The amount of cytokine production when T cells are stimulated with KLH.

FIG. 4E. The results after examining whether T cells can survive in vivo by using mice injected with SEA and/or LPS. The wild-type (◯) and OX40L transgenic mice (●) were injected with SEA, wild-type mice (□) and OX40L transgenic mice (▪) were injected with LPS and SEA.

FIG. 5 is a view that shows a polyclonal B cell activation in the OX40L transgenic mice of the present invention.

FIG. 5A. The “◯” in the figure shows the concentration of the serum immunoglobulin isotype in wild-type mice, “●” shows the concentration of the serum immunoglobulin isotype in OX40L transgenic mice, respectively.

FIG. 5B. The “◯” in the figure shows the serum anti-DNA antibody level against ssDNA or dsDNA in wild-type mice, “●” shows the serum anti-DNA antibody level against ssDNA or dsDNA in OX40L transgenic mice, respectively.

FIG. 5C. The “◯” in the figure shows the level of serum IL-5 or IL-13 in wild-type mice, “●” shows the level of serum IL-5 or IL-13 in OX40L transgenic mice.

FIG. 6 is a photograph that shows (A) the results of staining the lung, large intestine and lymph node of the OX40L transgenic mice and wild-type mice of the present invention with hematoxylin and eosin, and (B) the results of visualizing the CD4⁺ cells or CD8⁺ cells in the lung and large intestine of the OX40L transgenic mice of the present invention by fluorescence microscope.

FIG. 6A. The photograph on top shows the lung section magnified 200 times, the photograph in the middle shows the large intestine section magnified 100 times, and the photograph at the bottom shows lymph gland section magnified 1000 times, respectively.

FIG. 6B. The photograph on top shows the lung magnified 400 times, and the photograph at the bottom shows the large intestine magnified 200 times, respectively.

FIG. 7 is a photograph that shows (A) the results of staining the lung section and large intestine section in the rag-2-deficient mice transferred with the CD4⁺ T cells or CD8⁺ T cells derived from the OX40L transgenic mice of the present invention with hematoxylin and eosin, and (B) the results that the OX4OL transgenic mice of the present invention were treated with rat immunoglobulin or MGP34 monoclonal antibody, then the lung section and large intestine section in the rag-2-deficient mice introduced with the CD4⁺ T cells derived from the rat were stained with hematoxylin and eosin.

FIG. 7A. In the photograph, the lung section is magnified 200 times, and the large intestine section magnified 100 times, respectively.

FIG. 7B. In the photograph, the lung section is magnified 200 times, and the large intestine magnified 100 times, respectively.

### DETAILED DESCRIPTION OF THE INVENTION

In the present invention, in order to construct a transgenic non-human mammal that constantly expresses an OX40L (OX40 ligand) in the T cells, a non-human mammal can be used ad libitum. Non-human animals to be used in the present invention can include mice, rats, porcines, bovines, and other mammals. It is an embodiment of the present invention that for an effective use in such as screening for a therapeutic drug for an autoimmune disease, it is preferable to use mice. As for mice for constructing a transgenic non-human mammal, various kinds can be used, such as C57BL/6, DBA/2, etc. Transgenic mice can be constructed, for example, by introducing an expression vector comprised of an OX40L cDNA into (C57BL/6×DBA/2) F1 fertilized eggs. For the transgenic mice introduced with an OX40L gene, purification of the introduced gene is attempted by backcrossing the F1 mice confirmed to be introduced with the gene and parent mice. In the present invention, for example, backcross for 12 generations with the background of C57BL/6 parent mice is conducted. Additionally, other methods of creating transgenic animals may be used, as would be known to one of skill in the art.

The OX40L gene used in the present invention is publicly known, for example, it is registered as GenBank Accession No. U12763 (SEQ ID NO: 1). As for the expression vector used for the introduction of OX40L gene, a publicly known expression vector can be used. As for the promoter used for the expression vector, any ad libitum promoter may be used, as long as it is a vector having a function to constantly express OX40L in T cells. T cell-specific lck promoter can be given as an example of a preferable promoter.

Furthermore, it is also contemplated by the present invention that the OX40L gene can comprise a homologue, varient, derivative or fragment of the DNA sequence of Seq ID NO: 1, wherein the homologue, derivative, varient or fragment thereof has at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% homology to SEQ ID No: 1, or is complementary thereto.

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Alternatively, relative sequence identity can also be determined by commercially available computer programs that can calculate % identity between two or more sequences using any suitable algorithm for determining identity, using for example default parameters. A typical example of such a computer program is CLUSTAL. Other computer program methods to determine identify and similarity between the two sequences include but are not limited to the GCG program package (Devereux et al 1984 Nucleic Acids Research 12: 387) and FASTA (Atschul et al 1990 J Molec Biol 403-410).

The sequence identity or percent homology for proteins and nucleic acids can also be calculated as (Nref−Ndif)×100/Nref, wherein Ndif is the total number of non-identical residues in the two sequences when aligned and wherein Nref is the number of residues in one of the sequences. Hence, the DNA sequence AGTCAGTC will have a sequence identity of 75% with the sequence AATCAATC (Ndif=2 and Nref=8).

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (Ausubel et al., 1999 ibid , pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available at http://www.ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XN U program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs. It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST. In some embodiments of the present invention, no gap penalties are used when determining sequence identity.

The transgenic non-human mammal model of autoimmune disease introduced with an OX40L gene of the present invention can be used for screening a therapeutic drug for an autoimmune disease.

As for the method for screening, it can be conducted, for example, by administering a test substance to the mouse model having an onset of autoimmune disease, then evaluating and determining the symptoms of the autoimmune disease of the mouse model. As for the method for evaluating and determining the symptoms of the autoimmune disease, an example can be given wherein the pathological tissues are collected from the mouse administered with a test substance, then the tissue image is analyzed and evaluated. Furthermore, when determining/evaluating the degree of the development and/or progress of the symptom of autoimmune disease, it is preferable to simultaneously use a wild-type mouse of the same species as the mouse model of autoimmune disease development, as a precise comparative experiment can be conducted at individual level.

The therapeutic drug for an autoimmune disease obtained from the screening method of the present invention can be effectively applied to therapies such as interstitial pneumonia, inflammatory bowel disease, splenomegaly or lymphadenopathy, or hyperimmunoglobulinemia and the like, which develops as an autoimmune disease.

### EXAMPLES

The present invention will be explained more specifically with the following examples, however, the technical scope of the present invention will not be limited to these examples.

### Example 1

### Construction of OX40L Transgenic Mouse

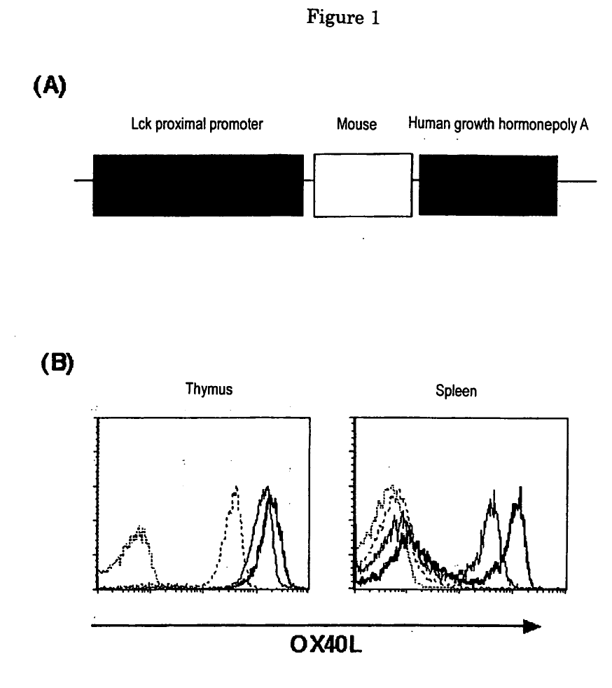

OX40L transgenic (OX40L-Tg) mouse was constructed by pronucleus injection of an expression plasmid DNA that encodes mouse OX40 ligand. The construct of the mouse OX40L expression vector was composed of mouse OX40L cDNA under the control of a T cell-specific lck promoter (FIG. 1A). Three first generation mice were constructed using Fl fertilized eggs (C57/BL/6×DBA/2) injected with OX40L expression plasmid DNA, then these mice were mated with C57BL/6 mice. Flow cytometry analysis showed OX40L expression also in the thymocyte group derived from the three OX40L-Tg strain mice, however, it has been found that such expression is not seen in those derived from wild-type mice (FIG. 1B). Among the three OX40L-Tg strain mice, 2 of the OX40L-Tg (OX40L-Tg1 and OX40L-Tg2) mice showed significant OX40L expression in the splenocytes, however, in the remaining 1 mouse (OX40L-Tg3), only weak expression of OX40L was observed (FIG. 1B). Therefore, the 3 strains of transgenic mice were backcrossed onto the C57BL/6 strain mice for at least 12 times.

### Example 2

### Spontaneous Activation of T Cells in OX40L Transgenic Mouse

First, flow cytometry was used to examine whether the OX40L-introduced gene, as a result, brings out any endogenous change in all the lymphoid cell groups in the thymus, spleen and lymph node. The number of thymus T cells and subgroups of the population examined by CD4⁺ and CD8⁺ expression did not show apparent abnormality in the 3 OX40L-Tg strains. However, the total number of lymphocytes in the spleen and lymph node increased significantly only in OX40L-Tg1 mice and OX40L-Tg2 mice. Especially, CD4⁺ T cells increased 2 fold, however, for CD8⁺ T cells, it was not detected also in these 2 OX40L-Tg strains (FIG. 2A). Only a slight change in the number of lymphocytes in the OX40L-Tg3 spleen or lymph node was observed. Considering the proliferation of the CD4⁺ T cell group in the OX40L-Tg1 mice, it was examined whether the mice retained T cells having activated phenotypes. The cell groups that express CD25 and CD69, which are activated T cell markers, significantly increased in spleen CD4⁺ T cells derived from OX40L-Tg1 mice, in contrast to wild-type mice (FIG. 2B). The same result was obtained when OX40L-Tg2 mice were used. These results revealed that many CD4⁺ T cells in the OX40L-Tg mice are spontaneously activated. In the B cell group, the number of IgM⁺ or B220⁺ B cells in the spleen hardly increased by the OX40L-introduced gene.

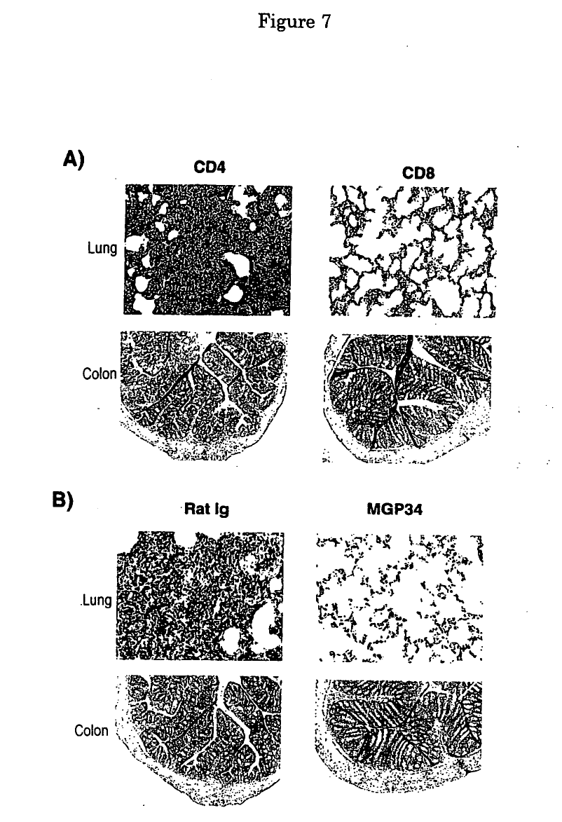

As there is a report that the OX40L/OX40 system is involved in the survival and maintenance of memory T cells (Gramaglia et al., 1998, J. Immunol., 161, 6510 and 2000, J. Immunol., 165, 3043), the memory T cell group in the OX40L-Tg1 mice was evaluated, and was compared with the case of wild-type and OX40L-deficient mice (Murata et al., 2000, J. Exp. Med., 191, 365). As a result, in the memory T cells derived from OX40L-Tg mice, the expression of CD44 increased, while the expression of CD62L and CD45RB decreased. The ratio (%) and number of CD62L^{low} CD44^{high} and CD45RB^{low} memory T cells increased significantly in the spleen CD4⁺ T cells of OX40L-Tg1 mice, in comparison to wild-type mice, while it was decreased in OX40L-deficient mice (FIGS. 3A and B). The increase of age-dependent T cells observed in wild-type mice was not observed in OX40L-Tg1 mice. The numerical increase of memory T cells was observed in these mice as early as 4 weeks of age, and it was shown that this continuous increase is not age-dependent (FIG. 3A). Based on these results, it was revealed that the emergence of the memory T cells in the OX40L-Tg mice was promoted. Next, the amount of cytokine production in these mice after administration of antigen was determined. As a result, the functional analysis of naive T cells and memory T cells was conducted. The naive (CD62L^{high} CD44^{low}) CD4⁺ T cells or (CD62L^{low} CD44^{high}) CD4⁺ T cells derived from OX40L-Tg1 mice or wild-type mice were stimulated with PMA and ionomycin. Then, cytoplasm IL-2, IFNγ and IL-4 were detected by immunostaining. Those derived from OX40L-Tg1 mice showed more significant increase than those derived from wild-type mice in both memory T cells and naive T cells, regarding all of the IL-2 positive cell group, IFNγ positive cell group and IL-4 positive cell group (FIG. 3C), and this indicates that the OX40 positive CD4⁺ T cells tend to activate a phenotype also in vivo. The increase of the memory T cells was confirmed by further analyzing by using Vβ of spleen T cells. These results revealed that most part of the peripheral T cells of OX40L-Tg is in the activated state and memorizing state.

### Example 3

### Increase of T Cell Activation in OX40L Transgenic Mouse

In order to evaluate the functional ability of memory T cells in the OX40L transgenic mice, several experiments to determine the proliferation and cytokine production ability in response to various stimuli of T cells was conducted. First, the proliferation reaction by phorbol myristate acetate (PMA) added with anti-CD3 antibody, concanavalin A (Con A) or ionomycin was evaluated regarding the spleen T cells derived from non-immunized wild-type mice and OX40L-Tg1 mice. The OX40L-Tg1 T cells showed a significantly higher proliferative reaction compared to the wild-type T cells, against all the stimuli (FIG. 4A). The ability of cytokine production of the T cells stimulated with anti-CD3 antibody was also confirmed by immunostaining. It was shown that the T cell groups that produce each of IL-2, IFNγ, IL-4, IL-5 and IL-10 increased significantly in the OX40L transgenic mice compared to the wild-type mice (FIG. 4B).

Next, an antigen-specific recall proliferation assay was conducted regarding the T cells derived from OX40L-Tg1 mice and wild-type mice. The T cells mentioned above were both treated with keyhole limpet hemocyanin (KLH) by complete Freund's adjuvant in vivo. The T cells of OX40L-Tg1 mice showed greater proliferative reaction compared to the wild-type mice (FIG. 4C). It was found that all the cytokine production of such as IL-2, IFNγ, IL-4, IL-5 and IL-10 from the T cells of the OX40L-Tg1 mice also increased significantly compared to the wild-type mice (FIG. 4D). These results conform to the previous report (Ndhlovu et al., 2001, J. Immunol., 167, 2991) of the present inventors by MOG stimulation, and show that the T cells of OX40L-Tg mice react to an antigen more efficiently compared to that of wild-type mice. In order to further examine the increase of memory T cells in OX40L-Tg mice, the survival ability in vivo was compared for the T cells of the OX40L-Tg1 mice and wild-type mice. The survival ability of the T cells in vivo was analyzed using mice wherein the SEA that activates T cells that express Vβ3 and the LPS known as a cell death-inducing activity inhibitor were inoculated (Maxwell et al., 2000, J. Immunol., 164, 107). In comparison to wild-type mice, the OX40L-Tg1 mice showed very little decrease of CD4⁺ T cells that express Vβ3 (FIG. 4E), and this indicates that the expression of OX40L on the T cells is involved in the suppression of T cell death-inducing activity in vivo.

### Example 4

### Activation of Polyclonal B Cells in OX40L Transgenic Mouse

Although increase in the number of B cells in the OX40L transgenic mice was not observed, concentration of serum immunoglobulin isotype was determined to examine whether the B cells are activated in vivo. In comparison to wild-type mice, the OX40L-Tg1 mice showed increase in level such as 10 fold for IgG1, 22 fold for IgE and 25 fold for IgA. On the other hand, increase of IgM, IgG2a and IgG2b was low, and they were 3 fold, 4 fold and 4 fold, respectively, in the sera of OX40L-Tg1 mice. Further, regarding the level of IgG3, the serum of OX40L-Tg1 mice was not higher than that of wild-type mice (FIG. 5A). This biased production of IgG isotype seen in OX40L-Tg1 mice can be explained by the increase of cytokine secretion level of the T cells as mentioned above. Moreover, it was confirmed that the serum anti-DNA antibody level against ssDNA and dsDNA increased significantly in the sera of OX40L-Tg1 mice (FIG. 5C). These results reveal that the OX40L-Tg1 mice activate the polyclonal antibody of B cells and lead to the production of autoimmunity. Further, the serum level of the various cytokines in the mice was analyzed. The level of IL-5 and IL-13 increased significantly in the sera of OX40L-Tg1 mice, compared to wild-type mice. However, the other cytokines such as IL-2, IL-4, IL-10, IFNγ and the like were not detected in either of OX40L transgenic mice or wild-type mice (FIG. 5B). IL-5 is involved in the increase of serum IgA level in the OX40L transgenic mice observed by the present inventors, and is thought to be its cause.

### Example 5

### Development of Autoimmune Inflammatory Disease in OX40L Transgenic Mouse

It was found by histological determination that autoimmune symptoms appeared in each organ of the OX40L transgenic mice. Significant invasion of lymphocytes at the bronchus and perivascular end portion and cholesterin-like crystal containing a great number of eosinophilic foam cells at the pulmonary alveolus were observed in lungs of the 9 month-old OX40L-Tg1 mice, and severe interstitial pneumonia was developed (FIG. 6A). Interstitial pneumonia was observed both in OX40L-Tg1 mice and OX40L-Tg2 mice in an early period of 3 months after birth (Table 1), indicating the possibility that the development of interstitial pneumonia is age-dependent.

TABLE 1

| | | | Number of mice with | |
|---|---|---|---|---|
| | | | autoimmune disease^{a} | |
| | | Number of | Interstitial | Inflammatory |
| Age (month) | Mice | mice | pneumonia | bowel disease |

a) Development of autoimmune diseases in
OX40L transgenic mice

| 6-7 weeks | Wild-type | 4 | 0^{b} | 0^{b} |
|---|---|---|---|---|
| | Tg | 9 | 4 (44%) | 3 (33%) |
| Less than 3 | Wild-type | 10 | 0 | 0 |
| months | Tg | 13 | 13 (100%) | 13 (100%) |
| | | | Number of mice with | |
| | | | autoimmune disease^{a} | |
| Inoculated | | Number of | Interstitial | Inflammatory |
| cells | Mice | mice | pneumonia | bowel disease |

b) Induction of autoimmune
diseases by T cell transfer

| | | | | |
|---|---|---|---|---|
| CD4⁺ T cells | Wild-type | 6 | 0^{b} | 0^{b} |
| | Tg | 11 | 11 (100%) | 11 (100%) |
| CD8⁺ T cells | Wild-type | 6 | 0 | 0 |
| | Tg | 3 | 0 | 0 |

^{a}Number of mice with a histopathologically evident autoimmune diseases
^{b}Incidence

The inflammatory bowel disease of the OX40L transgenic mice became clear by analysis of the intestinal tissues. In the 9 month-old OX40L-Tg1 mice, moderate to severe hyperplasia of lymphatic tissues in the intestinal basal membrane, hyperplasia of mucous epithelium in basal membrane, lymphocyte invasion or hyperplasia of submucous lymphoid follicle was observed (FIG. 6A). These inflammatory bowel diseases were observed both in OX40L-Tg1 mice and OX40L-Tg2 mice only in 3 months after birth (Table 1).

Splenomegaly and lymphadenopathy were observed in OX40L transgenic mice. Increase of plasmacyte and formation of Russell body was found in that lymph node. The Russell body reflects the deposit in the eosinophilic cells in the plasmacyte and the abnormal production of antibody, and is often accompanied with hyperimmunoglobulinemia (FIG. 6A). The other tissues of the OX40L transgenic mice such as heart, kidney and liver are histologically normal, and glucose and protein do not exist in the urine, therefore, evidence that shows the dysfunction of kidney or pancreas was not observed.

### Example 6

### Induction of Autoimmune Disease in RAG-2-Deficient Mice by Introduction of CD4⁺ T Cells of OX40L Transgenic Mouse

In order to confirm that the interstitial pneumonia of the OX40L-Tg mice is not due to infectious substance, a number of experiments that confirm the SPF condition of the mice were conducted. No evidence that the pneumonia and bowel disease of the mice are due to contagious microorganisms, could be found. In order to examine whether pathogenic autoreactive T cells are developed in the OX40L-Tg mice, CD4⁺ T cells and CD8⁺ T cells derived from Tg mice were transferred to C57BL/6 RAG-2-deficient mice that are deficient with T cells and B cells. In CD4⁺ T cells, the RAG-2-deficient mice showed weight loss and diarrhea-like symptom 3 week after the inoculation, however, nothing was indicated for CD8⁺ T cells (Table 1). In the histological analysis from after 2 weeks to 4 weeks from transfer of CD4⁺ T cells, a severe lymphoid interstitial pneumonia and large intestinal inflammatory bowel disease was observed (FIG. 7A). In either case where CD4⁺ T cells or CD8⁺ T cells derived from wild-type mice were inoculated, autoimmunity was not developed in RAG-2-deficient mice. Based on these results, it is shown that the CD4⁺ T cells derived from the OX40L transgenic mice is an important effector cell regarding the induction of autoimmune disease in RAG-2-deficient mice. The CD4⁺ T cells of the RAG-2-deficient mice transferred with said cells secreted a high level of IL-5 and IL-13, in the same manner as the CD4⁺ T cells of the OX40L-Tg mice. The induction of autoimmune disease in the lung and large intestine was suppressed by administration of MGP34, a monoclonal antibody that specifically inhibits against mouse OX40L, and by transfer of CD4⁺ T cells from OX40L-Tg mice to RAG-2-deficient mice (FIG. 7B). These results revealed that the OX40/OX40L interaction in the T cells is necessary for the development of lymphoid interstitial pneumonia and large intestinal inflammatory bowel disease.

(Evaluation)

It has been verified that the reaction of lymphocyte changes by the change of expression between several kinds of TNF family members. When Fas or its ligand, Fas ligand, is not present, a severe autoimmune disease is developed (Watanabe-Fukunaga et al., 1992, Nature, 356, 314; Takahashi et al., 1994, Cell, 76, 969). Further, it is thought that CD30 ligand has an action to prevent autoimmune diabetes. On the contrary, the increase of expression in BAFF or CD40 ligand, respectively, leads to the development of significant autoimmune disease and inflammation (Mackaw et al., 1999, J. Exp. Med., 190, 1697; Mehling et al., 2001, J. Exp. Med., 194, 615). Based on the results shown in the present Examples, it was revealed by the OX40/OX40L interaction that the stimulation of OX40 secretes autoimmune antibodies and develops inflammatory invasion in the lung and large intestine.

The present inventors have identified the OX40L (human gp34) ad initium as a molecule induced by a Tax gene of human T cell leukemia virus type I (HTLV-I) (Miura et al., 1991, Mol. Cell. Biol., 11, 1313). As the cultured normal human T cell clone expresses OX40L, an examination regarding the immune causation of OX40L that is involved in the OX40 signaling and is overexpressed on the T cells was conducted. The present inventors found that the spleen T cells and lymph node T cells of the OX40L transgenic mice express CD69 and CD25 at a high level and that over 60% of the CD4⁺ T cells are CD62L^{low}, CD44^{high} and CD45RB^{low}. This indicates that these T cell groups are activated memory phenotypes. These T cells were further analyzed, and surprisingly, it was found that the conversion to T cell memory phenotype is not age-dependent. In addition, it was revealed that the memory T cells derived from OX40L-Tg mice, when compared to the case of wild-type mice, increase the recall cytokine reaction after stimulation and enhance the antigen-dependent reaction. These initial findings conformed to the previous matters in question when clarifying the role of OX40/OX40L by prolongation of effector memory T cell reaction (EAE and the like). Further, it has been found that the long survival period of peripheral T cells stimulated with super antigens becomes longer in OX40L-Tg mice (FIG. 4E). The stimulation by OX40 induces initial increase and enhancement of durability after the stimulation by SEA and LPS (Maxwell et al., 2000. J. Immunol., 164, 107). It has been revealed that the spontaneous stimulation of OX40 enhances the antigen-dependent formation of the memory of T cells by control of a primary polyclonal extension. The results of the present Examples revealed that the OX40/OX40L signaling is involved in the conservation and survival of memory T cell groups in vivo.

OX40 is reported to express in autoreactive T cells of patients suffering from autoimmune disease such as rheumatoid arthritis, GVHD, lupus nephritis and the like, and mouse model of multiple sclerosis and EAE (Weinberg et al., 1999, J. Immunol., 162, 1818; Tittle et al., 1997, Blood, 89, 4652; Stuber et al., 1998, Gastroenterology, 115, 1205; Higgins et al., 1999, J. Immunol., 162, 486). In addition, it is described that the soluble OX40 fusion protein (antagonist of OX40L) which abolishes OX40/OX40L signaling suppresses progressing EAE (Weinberg et al., 1999, J. Immunol., 162, 1818; Ndhlovu et al., 2001, J. Immunol., 167, 2991), semiallogenic graft versus host disease (GVHD) (Stuber et al., 1998, Gastroenterology, 115, 1205), and improves progressing colitis in inflammatory bowel disease (IBD) mouse model (Higgins et al., 1999, J. Immunol., 162, 486). Based on the results mentioned above, it is considered that the OX40/OX40L interaction plays an important role in the immune control of various autoimmune diseases. The unexpected finding in the histological determination of the transgenic mice was the development of inflammatory disease in the lung and large intestine. In the interstitial tissue of the lung and basal membrane of the intestine of Tg mice, significant invasion was observed in CD4⁺ T cells, however, not in CD8⁺ T cells (FIG. 6B). The causal factor of autoimmune disease development of these mice was examined. Interstitial pneumonia and inflammatory bowel disease were developed by reconstruction of CD4⁺ T cells from OX40L-Tg mice to RAG-2-deficient mice, indicating that CD4⁺ T cells are important effector cells. With the CD4⁺ T cell transfer from OX40L-Tg mice to RAG-2-deficient mice, MGP34, a monoclonal antibody that specifically inhibits mouse OX40L was administered, and the histological change found previously in the lung was prevented (FIG. 7B). The symptom of the interstitial pneumonia in the OX40L-Tg mice is the same as the histological characteristic of human lymphatic interstitial pneumonia (LIP). Interestingly, abnormality of the lung was observed in patients of human T cell leukemia virus type 1 (HTLV-1)-related myelopathy (HAM/TSP) and HTLV-1-related uveitis and patients of HTLV-1 (Sugimoto et al., 1987, Lancet, 2, 1220; Maruyama et al., 1989, Medical Immunil., 18, 763; Setoguchi et al., 1991, Am. Rev. Res. Dis. 144, 1361; Sugimoto et al., 1997, Jpn. J. Chest Dis., 35, 184; Sugisaki et al., 1998, Am. J. Trop. Med. Hyg, 58, 721). Regarding the OX40L expression, those regarding the HTLV-1 infected human T cell line was described (Tanaka et al., 1985, Int. J. Cancer, 36, 549; Miura et al., 1991, Mol. Cell. Biol. 11, 1313) ad initium, and recently, those regarding cytotoxic T lymphocyte clone specific to EB virus infected B cell line are found (Takasawa et al., 2001, Jpn. J. Cancer Res., 92, 377). The expression of OX40L indicates the role in OX40/OX40L through the interaction between T cells, and is involved in the development of pulmonary disease found in these patients. Therefore, these mice are useful as a mouse model of interstitial pneumonia.

The expression of OX40/OX40L in the large intestine and jejunum of patients with inflammatory bowel disease (Crohn's disease and ulcerative colitis) are already known. Higgins et al. have indicated that when OX40L is inhibited with OX40-IgG fusion protein, the symptoms of hapten-induced colitis or the symptoms of IL-2 knockout mice suffering from idiopathic colitis improve. Hapten-induced colitis is often used as a model of human inflammatory colitis. The direct influence of hapten for the induction of colitis cannot be denied, however, it is impossible to show the pathogen of human inflammatory bowel disease in this model.

In other mouse models such as T cell reconstructive tgε26 mice, IL-7Tg mice, C.B-17scid mice, which are transgenic to IL-2^{-/-} mice, IL-2Rα^{-/-} mice, IL-10^{-/-} mice, TGFβ1^{-/-} mice, TCRα^{-/-} mice, Gαi2^{-/-} mice, human CD3ε gene, inflammatory bowel disease is developed (Sadlack B. et al., 1993, Cell, 75, 253; Kuhn R. et al., 1993, Cell 75, 263; Mombaerts P et al., 1993, Cell, 75, 275; Rudolph U et al., 1995, Nat. Genet. 10, 143; Hollander G A et al., 1995, Immunity, 3, 27; Watanabe et al., 1998, J. Exp. Med., 187, 389; Sundberg J P., 1994, Gastroenterology, 107, 1726; Powrie F et al., 1993, Int. Immunol., 5, 1461). Inflammatory bowel disease is a result of malfunction of immune control, and said disease is mainly mediated by the activation of CD4⁺ T cells. It has been confirmed by the results obtained presently by the present inventors, that the CD4⁺ T cells play an important role in the development of naturally developing colitis of mice. The spontaneous development of inflammatory bowel disease in the OX40L-Tg mice clarified the possibility that the OX40/OX40L interaction is involved in the cause of inflammatory bowel disease, and a feasible goal in the immunnotherapy of gastrointestinal diseases was obtained.

The basic structure of autoimmunity seen in Tg mice can be explained by recent studies which showed that the antagonist OX40 antibody can disrupt the tolerance of T cells, however, the role of cytokines remain to be elucidated. OX40L-Tg mice suffering from naturally developing pneumonia and bowel disease showed increase in the level of Th-2 cytokine, IL-5 and IL-13 in the sera (FIG. 5C). The same increase in the level of cytokine was observed also in Rag-2-deficient mice wherein CD4⁺ T cells derived from OX40L-Tg mice are reconstructed. It was found that OX40L-Tg mice are Th-2 dominant. The involvement of Th-1/Th-2 cytokine to the development of interstitial pneumonia is indicated. IL-4 transgenic mice and IL-13 transgenic mice induce an inflammatory reaction characterized by lung mononuclear cells, hyperplasia of airway epithelium, excess of mucous secretion and hyperplasia of goblet cells (Zhu et al., 1999, J. Clin. Inv. 103, 779). IL-5 in the bronchoalveolar lavage showed high level in patients with pneumonia (Taniguchi et al., 2000, Eur. Respir. J., 16, 959). There are cases when the high production of IL-13 and IL-5 mice is directly involved in the development of interstitial pneumonia in OX40L-Tg mice. It becomes possible to induce lymphatic interstitial pneumonia and inflammatory bowel disease of the large intestine by transfer of CD4TgT lymphocytes to Rag-2-deficient mice, and it is shown that this is a disease mediated by CD4⁺ T cell-dependent substance.

Recently, OX40 is emphasized as a main co-stimulating molecule that can activate T cells. OX40-deficient mice or OX40L-deficient mice could not support the strong CD4⁺ T cell reaction in vivo (Chen et al., 1999, Immunity, 11, 689; Pippig et al., 1999, J. Immunol., 163, 652; Murata et al., 2000, J. Exp. Med., 191, 365). Further, an antigen was given to the OX40L-deficient mice, and it was observed that the antigen presenting cell function was disrupted (Murata et al., 2000, J. Exp. Med., 191, 365). It was found out from these data that OX40/OX40L interaction is indispensable to the T-APC interaction in vivo. The present inventors constructed mice that show constructive expression of OX40L on the T cells and constantly stimulated OX40, and eventually, clarified the further importance of OX40L in the memory development of T cells, to the series of phenomena which are to become an organ-specific autoimmune disease.

(Method 1 Construction of OX40L-Tg Mouse)

T cell line-specific vector p1017 (a vector wherein a poly A signal derived from human growth hormone gene is integrated to lck promoter; obtained from Perlmuttaer) was introduced with a cDNA that encodes mouse OX40 ligand. Said introduced gene was microinjected into the pronucleus of F1 fertilized egg (C57BL/6×DBA/2), the ova obtained were cultured, then transplanted into the oviduct of foster parent mice to produce baby mice. From the obtained baby mice, first generation mice having the introduced gene mentioned above were identified by PCR, and for further experiment, said first generation mice were further backcrossed for at least 12 times with the C57BL/6 strain mice. Before the injection of mouse fertilized eggs, the ability was confirmed by introducing the gene mentioned above into the mouse culture cells. The introduced gene was introduced into the T cell line by electroporation method, and a high level of surface OX40 ligand protein was detected.

(Method 2 FACS Analysis)

In order to remove the cells that associate non-specifically to the labeled monoclonal antibody from among the cell group containing those that bind to Fc receptor, preincubation was conducted using normal rat sera, and incubation was conducted for 30 minutes at 4° C. using a labeled monoclonal antibody. After the incubation, the specimen was washed and analyzed with a FACSCalibur flow cytometer (Becton Dickinson). For analysis, CELLquest (Becton Dickinson) analysis software was used. CD3, CD8, CD4, CD5, CD25, CD69, B220, IgM, CD44, CD45RB, IL-2, IL-4 and IFNγ were purchased from Pharmingen. MGP34 (IgG2c) specific to mouse OX40L in the cells established by the method above and MOX40 (IgG1) specific to mouse OX40 were bound to NHS-LC-biotin (Pierce Chemical Co.). The cells labeled with biotinylated monoclonal antibody were visualized with streptavidin-APC (Pharmingen), then analyzed with flow cytometry. For OX40L or OX40 negative control, the unlabeled MGP34 and MOX40 were used, respectively, to preincubate the cells mentioned above, and those that were specifically stained by biotinylated antibody were invalidated.

In order to stain the intracellular cytokine, single cell suspension of spleen or lymph node cells was stimulated for 2 hours with 50 ng/ml PMA (Sigma) and 1 μM ionomycin (Sigma), then Gorgi Stop (Pharmingen) was added and further cultured for 2 hours. After the stimulation, the cells were stained with APC-labeled CD4 or CD8, then the cells were fixed, penetration treatment was conducted with Cytofix/Cytoperm kit (Pharmingen). Further, the cells were stained by the method recommended by the manufacturer, according to the protocol of the manufacturer by using PE labeled anti-IL-2 antibody, anti-IL-4 antibody or anti-IFN-γ antibody.

(Method 3 Purification and Culture of Cells)

CD4⁺ T cells or CD8⁺ T cells were concentrated from spleen cells or lymph node cells concentrated by magnetic beads or separated by Auto MACS (Miltenyi Biotec). The T cells (1×10⁵) obtained from 6 week-old wild-type mice or OX40L-Tg mice were cultured in a medium only, or in a medium added with Con A (10 μg/ml), PMA (10 ng/ml) added with ionomycin (1 μg/ml), or fixed anti-CD3 monoclonal antibody (10 μg/ml), in order to grow the T cells, for 48 hours, respectively, and the intake amount of each ³H thymidine was analyzed.

(Method 4 Priming Reaction and Recall Reaction of T Cells by Protein Antigen Stimulation in Vivo)

OX40L transgenic mice or wild-type littermate mice were injected with 100 μg KLH together with complete Freund's adjuvant to the pads of the posterior limb, respectively. 9 days later, the lymph node cells were incubated for 3 days at 37° C. together with KLH at the concentration described in the Figure. Meanwhile, the CD4⁺ T cells purified from the lymph node was stimulated in the same manner in the presence of APC by using KLH. Before use, said APC was isolated from the spleen of the wild-type littermate mice, and treated with radiation (3,000 rad). The cultured cells mentioned above were analyzed regarding the uptake of ³H thymidine and cytokine production in the reaction against KLH in vitro, in the same manner as previously described (Takeshita et al., 1989, J. Exp. Med., 169, 1323; Nagata et al., 1999, J. Immunol., 162, 1278). In the cytokine production, from the second addition of KLH, 48 hours after for IL-2 or IL-4, 96 hours after for IL-5, IL-10 or IFNγ, the supernatant of the culture solution was collected. Each supernatant was subjected to ELISA, and the cytokine production was measured.

(Method 5 ELISA)

The cytokine levels in the supernatant solutions of tissue cultures or mouse sera were analyzed by ELISA according to the protocol of the manufacturer, using an antibody against IL-2, IL-4, IL-5, IL-10, IL-13 or IFNγ (Pharmingen).

(Method 6 Secretion of Immunoglobulin)

The level of the various immunoglobulin subclasses in the mouse sera was analyzed. Coating was conducted by incubating each well of the ELISA microplate overnight at 4° C., with a carbonic buffer including 10 μg/ml goat anti-mouse immunoglobulin antibody. After washing the plates, the goat anti-mouse immunoglobulin antibody was blocked for 1 hour at 37° C., with PBS containing 1% BSA. Sera derived from OX40L-Tg mice or wild-type mice diluted with PBS including 1% BSA were added to a well, and incubation was conducted at room temperature for 2 hours. After the plate was washed, the antibody detected by incubation was bound with goat anti-mouse IgM antibody, IgG1 antibody, IgG2a antibody, IgG2b antibody, IgG3 antobody or IgA antibody bond with alkali phosphatase (AP) (Southern Biotechnology Associates), and incubation was conducted for 1 hour. After the incubation, diethanol amine buffer including AP substrate (Sigma Chemical) was used for staining, then the reaction was stopped by 3 M NaOH, and the solution was evaluated with OD405 nm.

(Method 7 Histological and Immunohistological Analysis)

Tissues obtained from animals were fixed with 10% buffer formalin (Sigma), embedded with paraffin, then 5 micrometer of section was stained with hematoxylin and eosin by conventional method. Said tissue samples were embedded with OCT compound and then frozen, or frozen by liquid nitrogen, and stored at −80° C. Then, according to the protocol of the manufacturer, anti-mouse CD4 antibody (H129.19, Becton Dickinson) and anti-mouse CD8 antibody (53-6.7, Becton Dickinson) and FITC-labeled anti-rat IgG antibody (Seikagaku Corporation) were used to conduct immunohistochemical staining.

The invention will now be further described by the following numbered paragraphs:
1. A transgenic non-human mammal introduced with an OX40L gene and constantly expresses OX40L in T cells.
2. The transgenic non-human mammal according to paragraph 1, wherein the OX40L gene is comprised of a DNA sequence of GenBank Accession No. U12763.
3. The transgenic non-human mammal according to paragraph 1 or 2, wherein the OX40L gene is introduced under the control of a T cell-specific lck promoter.
4. The transgenic non-human mammal according to any one of paragraphs 1 to 3 introduced with the OX40L gene and has an onset of an autoimmune disease.
5. The transgenic non-human mammal according to paragraph 4, wherein the autoimmune disease is an interstitial pneumonia.
6. The transgenic non-human mammal according to paragraph 4, wherein the autoimmune disease is an inflammatory bowel disease.
7. The transgenic non-human mammal according to paragraph 6, wherein the inflammatory bowel disease has an onset of moderate to severe hyperplasia of lymphatic system in the intestinal basal membrane, hyperplasia of mucous epithelium in basal membrane, lymphocyte invasion or hyperplasia of submucous lymphoid follicle.
8. The transgenic non-human mammal according to paragraph 4, wherein the autoimmune disease is a splenomegaly or lymphadenopathy.
9. The transgenic non-human mammal according to paragraph 4, wherein the autoimmune disease is a hyperimmunoglobulinemia.
10. The transgenic non-human mammal according to any one of paragraphs 1 to 9, wherein the transgenic non-human mammal is a mouse.
11. A method for constructing a transgenic non-human mammal, wherein an expression plasmid DNA that encodes OX40L is injected into a pronucleus of a fertilized egg of a non-human mammal to introduce an OX40L gene into a non-human mammal.
12. The method for constructing a transgenic non-human mammal according to paragraph 11, wherein the OX40L gene is comprised of a DNA sequence of GenBank Accession No. U12763.
13. The method for constructing a transgenic non-human mammal according to paragraph 11 or 12, wherein the expression plasmid DNA that encodes OX40L is comprised of an OX40L cDNA under the control of a T cell-specific lck promoter.
14. The method for constructing a transgenic non-human mammal according to any one of paragraphs 11 to 13, wherein the transgenic non-human mammal introduced with an OX40L gene is purified by backcross.
15. The method for constructing a transgenic non-human mammal according to paragraph 14, wherein the backcross is conducted at least for 12 generations.
16. The method for constructing a transgenic non-human mammal according to any one of paragraphs 11 to 15, wherein the non-human mammal is a mouse.
17. The method for constructing a transgenic non-human mammal according to paragraph 16, wherein an OX40L expression vector wherein an OX40L gene is integrated downstream of the lck promoter, introduced into a mouse fertilized egg, and the mouse is backcrossed to a C57BL/6 line mouse.
18. A method for screening a therapeutic drug for an autoimmune disease that uses a transgenic non-human mammal according to any one of paragraphs 1 to 9.
19. The method for screening a therapeutic drug for an autoimmune disease according to paragraph 18, wherein the autoimmune disease is interstitial pneumonia, inflammatory bowel disease, splenomegaly or lymphadenopathy, or hyperimmunoglobulinemia.
20. The method for screening a therapeutic drug for an autoimmune disease according to paragraph 17 or 18, wherein a test substance is administered to the transgenic non-human mammal according to any one of paragraphs 1 to 9, and changes in symptoms of autoimmune disease are evaluated and determined.
21. The method for screening a therapeutic drug for an autoimmune disease according to any one of paragraphs 18 to 20, wherein the transgenic non-human mammal is a mouse.
22. A therapeutic drug for diabetes obtained from the method for screening a therapeutic drug for an autoimmune disease according to any one of paragraphs 18 to 21.

### INDUSTRIAL APPLICABILITY

The transgenic non-human mammal of the present invention constantly expresses an OX40 ligand in the T cells and develops an autoimmune disease, therefore, it is useful as an autoimmune disease model. The transgenic non-human mammal can be used effectively for screening a therapeutic drug for an autoimmune disease.

1. A transgenic non-human mammal introduced with an OX40L gene which constantly expresses OX40L in T cells.

2. The transgenic non-human mammal according to claim 1, wherein the OX40L gene is comprised of a DNA sequence of GenBank Accession No. U12763.

3. The transgenic non-human mammal according to claim 1, wherein the OX40L gene is introduced under the control of a T cell-specific lck promoter.

4. The transgenic non-human mammal according to claim 1 introduced with the OX40L gene and has an onset of an autoimmune disease.

5. The transgenic non-human mammal according to claim 4, wherein the autoimmune disease is an interstitial pneumonia.

6. The transgenic non-human mammal according to claim 4, wherein the autoimmune disease is an inflammatory bowel disease.

7. The transgenic non-human mammal according to claim 6, wherein the inflammatory bowel disease has an onset of moderate to severe hyperplasia of lymphatic system in the intestinal basal membrane, hyperplasia of mucous epithelium in basal membrane, lymphocyte invasion or hyperplasia of submucous lymphoid follicle.

8. The transgenic non-human mammal according to claim 4, wherein the autoimmune disease is a splenomegaly or lymphadenopathy.

9. The transgenic non-human mammal according to claim 4, wherein the autoimmune disease is a hyperimmunoglobulinemia.

10. The transgenic non-human mammal according to claim 1, wherein the transgenic non-human mammal is a mouse.

11. A method for constructing a transgenic non-human mammal, wherein an expression plasmid DNA that encodes OX40L is injected into a pronucleus of a fertilized egg of a non-human mammal to introduce an OX40L gene into a non-human mammal.

12. The method for constructing a transgenic non-human mammal according to claim 11, wherein the OX40L gene is comprised of a DNA sequence of GenBank Accession No. U12763.

13. The method for constructing a transgenic non-human mammal according to claim 11, wherein the expression plasmid DNA that encodes OX40L is comprised of an OX40L cDNA under the control of a T cell-specific lck promoter.

14. The method for constructing a transgenic non-human mammal according to claim 11, wherein the transgenic non-human mammal introduced with an OX40L gene is purified by backcross.

15. The method for constructing a transgenic non-human mammal according to claim 14, wherein the backcross is conducted at least for 12 generations.

16. The method for constructing a transgenic non-human mammal according to claim 11, wherein the non-human mammal is a mouse.

17. The method for constructing a transgenic non-human mammal according to claim 16, wherein an OX40L expression vector wherein an OX40L gene is integrated downstream of the lck promoter, introduced into a mouse fertilized egg, and the mouse is backcrossed to a C57BL/6 line mouse.

18. A method for screening a therapeutic drug for an autoimmune disease that uses a transgenic non-human mammal according to claim 1.

19. The method for screening a therapeutic drug for an autoimmune disease according to claim 18, wherein the autoimmune disease is interstitial pneumonia, inflammatory bowel disease, splenomegaly or lymphadenopathy, or hyperimmunoglobulinemia.

20. The method for screening a therapeutic drug for an autoimmune disease according to claim 17, wherein a test substance is administered to a transgenic non-human mammal introduced with an OX40L gene which constantly expresses OX40L in T cells, and changes in symptoms of autoimmune disease are evaluated and determined.

21. The method for screening a therapeutic drug for an autoimmune disease according to claim 18, wherein the transgenic non-human mammal is a mouse.

22. A therapeutic drug for diabetes obtained from the method for screening a therapeutic drug for an autoimmune disease according to claim 18.


SEQUENCE LISTING (1 sequence)

SEQ ID NO: 1 - DNA, 1633 bp, Mus musculus
agatcttggaacgagacgacctgctgggacctttatcttctgacccgcaggcttgactttgcccttattggctcctttgtggtgaagagcagtcttcccccaggttccccgccacagctgtatctcctctgcaccccgactgcagagatggaaggggaaggggttcaacccctggatgagaatctggaaaacggatcaaggccaagattcaagtggaagaagacgctaaggctggtggtctctgggatcaagggagcagggatgcttctgtgcttcatctatgtctgcctgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgtgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtgaattccactctgagggtggacgggacacaggttctttctcgagagagatgagtgcatcctgctcatgagatgtgactgaatgcagagcctaccctacttcctcactcagggatatttaaatcatgtcttacataacagttgacctctcattcccaggattgccttgagcctgctaagagctgttctgggaatgaaaaaaaaataaatgtctcttcaagacacattgcttctgtcggtcagaagctcatcgtaataaacatctgccactgaaaatggcgcttgattgctatcttctagaattttgatgttgtcaaaagaaagcaaaacatggaaagggtggtgtccaccagccagtaggagctggagtgctctctccaggttaaggtgatagaagtttacatgttgcctaaaactgtctctcatctcatggggggcttggaaagaagattaccccgtggaaagcaggacttgaagatgactgtttaagcaacaaggtgcactcttttcctggcccctgaatacacataaaagacaacttccttcaaagaactacctagggactatgatacccaccaaagaaccacgtcagcgatgcaaagaaaaccaggagagctttgtttattttgcagagtatacgagagatttttaccctgagggctatttttattatacagaatgatagtgaactggatgtctcaggataaaggccaagaaggatttttcacagtctgagcaagactgtttttgtaggtttctctctccaaaacttttaggtaaatttttgataatttttaaatttttatatttttggaccattttcaatagaagattgaaacatttccagatggtttcatatccccacaagagcaggtcgtctcgttccaagatct